(19) United States
(12) Patent Application Publication
(10) Pub. No.: US 2026/0194465 A1
(43) Pub. Date: Jul. 9, 2026

(54) OPTICALLY DETECTED MAGNETIC RESONANCE WITH LIGHT-SHEET MICROSCOPY

(71) Applicant: The Chinese University of Hong Kong, Hong Kong (CN)

(72) Inventors: Renbao LIU, Hong Kong (CN); Quan LI, Hong Kong (CN); Shuo WANG, Hong Kong (CN); Jingwei FAN, Hong Kong (CN); Mingzhong AI, Hong Kong (CN)

(21) Appl. No.: 19/475,701

(22) PCT Filed: Apr. 30, 2025
(86) PCT No.: PCT/CN2025/092357

§ 371 Date: Oct. 15, 2025

Related U.S. Application Data
(60) Provisional application No. 63/641,702, filed on May 2, 2024.

(51) Int. Cl. G01N 21/64; G01N 33/00; G02B 21/00; G02B 21/36

(52) U.S. Cl. CPC G01N 21/6458; G01N 33/389; G01N 33/39; G02B 21/0064; G02B 21/0076; G02B 21/36; G01N 2201/0231; G01N 2201/0238; G01N 2201/06113

(57) ABSTRACT

A system and methods based on light sheet microscopy (LSM) and optically detected magnetic resonance (ODMR) are provided. The system includes a cylindrical lens; an objective lens combined with the cylindrical lens to generate a light sheet for exciting a sample from one side; a collection objective lens for collecting fluorescence generated by the sample; a galvo mirror for scanning the light sheet to realize three-dimensional imaging and sensing; an antenna for introducing microwave with frequency sweeping to the sample; and an image sensor for collecting the fluorescence of the sample under each microwave frequency point with same exposure time to obtain ODMR signals. The sample is fixed on a 3D stage and the position of the sample is adjustable. The temperature sensitivity using the LSM-ODMR method can reach a few K/\sqrt{Hz}.

### CROSS-REFERENCE TO RELATED APPLICATION

The present application claims the benefit of U.S. Provisional Application Ser. No. 63/641,702, filed May 2, 2024, which is hereby incorporated by reference herein in its entirety, including any figures, tables, or drawings.

### BACKGROUND OF THE INVENTION

Nitrogen-vacancy (NV) centers are promising quantum sensors for biological studies for their good spin coherence under ambient conditions and chemical inertness [1-3]. The ground state of a negatively charged NV center is a spin triplet with magnetic quantum number mₛ=0, ±1. The NV center spin can be optically polarized to the mₛ=0 state by a green laser (for example, with wavelength 532 nm). The NV centers in the mₛ=0 state have brighter fluorescence (by about 30%) than those in the mₛ=±1 states, which makes fluorescence readout of the spin state possible. A microwave resonant with the spin transitions can rotate the spin between the mₛ=0 and mₛ=±1 states. If a frequency-sweeping microwave is added, the NV center can be driven to mₛ=1 states at the resonant frequency, leading to a fluorescence decrease. This is called optically detected magnetic resonance (ODMR) measurement [4]. The ODMR spectrum is sensitive to magnetic field [5], temperature [6,7], electric field [8], pressure [9], and many other parameters, making NV center a promising quantum sensor [10]. There are also detecting methods based on T₁ relaxation measurement of NV centers. The NV center spin is coupled with the surrounding environment and will lose its polarization, reaches thermal equilibrium finally. The time scale of population loss is called T₁. The NV center is firstly polarized to mₛ=0 state by a green laser pulse. After a varying dark time τ to evolve, the spin is optically read out by another green laser pulse. For the relaxation process from mₛ=±1 state, a π pulse is added after the first green laser pulse to flip the spin to mₛ=±1 state. Experimentally, to exclude other influences such as the change of the charge state of NV centers, T₁ relaxation curve is usually extracted by subtracting the fluorescence from mₛ=0 and mₛ=±1 state. The spin noise in the environment will accelerate the depolarization rate of NV centers so shortens T₁, allowing for using the variation of T₁ to detect paramagnetic spins in the environment, such as paramagnetic ions and reactive oxygen species [11-14]. Nanodiamonds (NDs) containing NV centers are ideal biosensors for their high material and fluorescence stability, good biocompatibility, sub-micron spatial resolution, and rich surface functionalization [1-3,15,16]. General NV center-based biosensing applications include nano thermometry in bio-systems [3,16-18], intracellular orientation tracking [19,20], sub-cellular magnetic imaging [21], physiologically relevant species sensing [11-14], etc.

However, the ODMR measurement, which is the key to NV center-based bio-sensing, requires laser irradiation, which will inevitably cause phototoxicity to bio-samples. Phototoxicity refers to photo-induced damage of life systems such as cells [22]. The origin of phototoxicity includes the generation of reactive oxygen species (ROS) [23], the chemical alteration and breakdown of cellular molecules [24], and thermal damage [25]. These effects accumulate and eventually lead to unrecoverable damage, until apoptosis. Thus, the phototoxicity has been a bottleneck issue for bio-imaging and bio-sensing in cells.

Confocal ODMR, in which the laser beam is concentrated at a single point (which usually has a lateral radius of several hundred nanometers in the xy plane and 2-3 times larger in the z axis), collects fluorescence within this point at a time. However, this approach imposes significant limitations on imaging speed and information retrieval. Obtaining a complete image of a bio-sample (by scanning the focused point across the entire sample) needs a high laser dose.

Compared to the confocal ODMR, widefield ODMR is useful in bio-imaging due to its high imaging speed and the possibility of correlating ODMR signals with spatial information. Nevertheless, the phototoxicity generated is higher than that from confocal ODMR because of the higher laser dose. The widefield illumination in the total-internal-reflection-fluorescence (TIRF) mode suppresses the phototoxicity by confining the light within the thin evanescent-wave layer at the basal surface [21,26,27]. However, it can only sense one thin layer of samples close to the surface (usually about 100 nm) [27] and is complicated by the interference of surface effects with the cellular functions, when dealing with bio-samples.

The light sheet microscopy (LSM) uses a thin, movable light layer to illuminate the sample perpendicular to the direction of observation [25-28]. The laser beam is focused only in one direction to form a light sheet [25]. Compared to the traditional microscopy, only the observed thin layer of sample is illuminated. Therefore, the optical sectioning ability of LSM can significantly reduce the phototoxicity [28-31]. When compared with the confocal microscopy, the LSM has much lower phototoxicity because every part of the specimen is only illuminated once (a minimum illumination) and has much higher imaging speed. When compared with the traditional widefield microscopy, the LSM eliminates the off-focus illumination (hence suppressed phototoxicity) and the off-focus fluorescence background (hence enhanced signal-to-noise ratio). When compared with the TIRF mode microscopy, the light sheet is movable in the z direction so the sensing ability is not confined to the evanescent layer and can realize 3D imaging of the sample. The comparisons of different excitation methods are shown in FIGS. 1A-1D.

There are several kinds of light sheets according to the type of beams. A Gaussian light sheet is formed by a cylindrical lens and an objective [28]. A parallel beam is focused in one direction after the cylindrical lens and the objective. The thickness of a Gaussian light sheet (2ω₀) is determined by the numerical aperture (NA) of the exciting lens and the wavelength of the light (λ) as

$$2 {ω}_{0} \approx 2 \frac{λ}{π \cdot NA} .$$

The length of the Gaussian light sheet 2zᵣ (where zᵣ is the Rayleigh length) is

$$2 {z}_{r} = 2 \frac{{πω}_{0}^{2} n}{λ}$$

Here n is the refractive index of the medium. The thickness and length of a Gaussian light sheet are constrained by the diffraction limit [28]. A thinner light sheet results in a shorter length. To overcome this problem, some other beams are used such as Bessel beam and Airy beam. The Bessel light sheet has a larger field of view but suffers from side lobes which will cause extra phototoxicity and out of focus fluorescence [30-32]. The Airy light sheet with an extended field of view has also been demonstrated [33].

### BRIEF SUMMARY OF THE INVENTION

Embodiments of the subject invention pertain to a method and systems of optically detected magnetic resonance with light-sheet microscopy.

According to an embodiment of the subject invention, a system based on light sheet microscopy (LSM) and optically detected magnetic resonance (ODMR) is provided. The system comprises a cylindrical lens CL; an objective lens O1 combined with the cylindrical lens CL to generate a light sheet for exciting a sample from one side; an acousto-optic modulator (AOM) to gate the exciting light; a collection objective lens O2 for collecting fluorescence generated by the sample; a galvo mirror for scanning the light sheet in a first direction to realize three-dimensional (3D) imaging and sensing; a signal generator to generate microwave; an RF switch to gate the microwave; a microwave amplifier to amplify the microwave; an antenna for introducing microwave with frequency sweeping to the sample; an image sensor for collecting the fluorescence of the sample under each microwave frequency point with same exposure time to obtain ODMR signals; and a pulse streamer to send pulse sequences to AOM, RF switch and the image sensor; wherein the sample is fixed on a 3D stage and a position of the sample is adjustable, and wherein the cylindrical lens CL and the objective lens O1 are fixed on a stage along a second direction which is a propagation direction of a laser beam such that a position of the light sheet is adjustable along the second direction. The collection objective lens O2 is disposed in a direction perpendicular to the objective lens O1. The system further comprises one or more filters for collecting the fluorescence in a predetermined wavelength range. Moreover, the image sensor is a sCMOS camera. The sample is a plurality of nanodiamonds. By scanning the light sheet in the first direction, ODMR spectra of the sample in a 3D volume are collected. Furthermore, a temperature sensitivity of the sample is determined and is on a scale of K/\sqrt{Hz}. The T₁ values of nanodiamonds in a 3D volume can also be collected. The 3D image of Hela cells and intracellular nanodiamonds is realized. The temperature sensitivity of intracellular nanodiamonds is on a scale of K/\sqrt{Hz}. The objective lens O₁ is an air immersion objective lens. The objective lens O₁ has a working distance (WD) of 20.3 mm and a numerical aperture (NA) of 0.21. The collection objective lens O2 is a water immersion objective lens having a working distance of 2.8 mm and a numerical aperture of 1.0.

In another embodiment of the subject invention, a method based on light sheet microscopy (LSM) and optically detected magnetic resonance (ODMR) is provided. The method comprises exciting a sample from one side, by a light sheet generated by a cylindrical lens CL and an objective lens O1; gating the exciting light sheet by an AOM; collecting, by a collection objective lens O2, fluorescence generated by the sample; scanning, by a galvo mirror, the light sheet in a first direction to realize three-dimensional (3D) imaging and sensing; amplifying microwave, by a microwave amplifier; gating the microwave, by an RF switch; introducing, by an antenna, the microwave with frequency sweeping to the sample; and an image sensor for collecting the fluorescence of the sample under each microwave frequency point with same exposure time to obtain ODMR signals; wherein the sample is fixed on a 3D stage and a position of the sample is adjustable, and wherein the cylindrical lens CL and the objective lens O1 are fixed on a stage along a second direction which is a propagation direction of a laser beam such that a position of the light sheet is adjustable along the second direction. The collection objective lens O2 is disposed in a direction perpendicular to the objective lens O1. The method further comprising providing one or more filters for collecting the fluorescence in a predetermined wavelength range. Moreover, the image sensor is a sCMOS camera. The sample is a plurality of nanodiamonds. By scanning the light sheet in the first direction, ODMR spectra of the sample in a 3D volume are collected. Furthermore, a temperature sensitivity of the sample is determined and is on a scale of K/\sqrt{Hz}. The T₁ values of nanodiamonds in a 3D volume can also be collected. The 3D image of Hela cells and intracellular nanodiamonds is realized. The temperature sensitivity of intracellular nanodiamonds is on a scale of K/\sqrt{Hz}. The objective lens O₁ is an air immersion objective lens. The objective lens O₁ has a working distance (WD) of 20.3 mm and a numerical aperture (NA) of 0.21. The collection objective lens O2 is a water immersion objective lens having a working distance of 2.8 mm and a numerical aperture of 1.0.

### BRIEF DESCRIPTION OF THE DRAWINGS

FIGS. 1A-1D show schematic representations of different excitation methods, including confocal microscopy in FIG. 1A; widefield microscopy in FIG. 1B; TIRF microscopy in FIG. 1C; and LSM in FIG. 1D.

FIG. 2 is a schematic representation of the LSM-ODMR setup, wherein L₁ to L₆ are convex lenses, AOM is an Acousto-optic modulator, Mi is a mirror, DM is a dichroic mirror, CL is a cylindrical lens, O₁ is the exciting objective, O₂ is the collecting objective, and L₇ is a tube lens.

FIGS. 3A-3B show the design of LSM-ODMR sample holders.

FIG. 4 is the visualization of the light sheet, wherein the larger figure shows the fluorescence intensity distribution of Rhodamine B solution illuminated by the light sheet, and the inset shows the thickness of the light sheet (about 1.6 μm at full width at half maximum).

FIGS. 5A-5B show representative results of the LSM-ODMR test, wherein FIG. 5A shows a typical ODMR spectrum and FIG. 5B presents a corresponding temperature sensitivity about 3 K/\sqrt{Hz}.

FIG. 6 shows the 3D distribution of NDs in agarose, wherein the x direction is the propagation direction of the laser, and the z direction is the scanning direction of the light sheet.

FIG. 7 shows the temperature sensitivity distribution of the 36 used NDs.

FIG. 8 shows exemplary dD/dT fitting results of the NDs, wherein x coordinate is the temperature measured by the thermocouple and y coordinate is the zero-field splitting D of the NDs.

FIG. 9 shows the distribution of dD/dT of the 36 used NDs.

FIG. 10 shows results of the temperature sensing experiment, wherein the box plots show the temperature detected by the NDs and the red points show the set temperature points.

FIGS. 11A-11B show the pulse sequences used for LSM-T₁ relaxation measurement (FIG. 11A) and LSM-Rabi oscillation (FIG. 11B).

FIGS. 12A-12B show representative results of the LSM-T₁ test (FIG. 12A) and Rabi oscillation (FIG. 12B).

FIGS. 13A-13B show images of the Hela cells and NDs using LSM-ODMR setup. FIG. 13A is the 3D image. FIG. 13B shows the x-y, x-z, y-z cross-sectional images.

FIGS. 14A-14B show the temperature sensitivity distribution of the nanodiamonds in a Hela cell using LSM-ODMR. FIG. 14A shows the image of the single Hela cell and the NDs. FIG. 14B shows the temperature sensitivity distribution of the NDs in FIG. 14A.

### DETAILED DISCLOSURE OF THE INVENTION

Embodiments of the subject invention are directed to a system and methods based on light sheet microscopy (LSM) and optically detected magnetic resonance (ODMR).

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention pertains. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

When the term “about” is used herein, in conjunction with a numerical value, it is understood that the value can be in a range of 90% of the value to 110% of the value, i.e. the value can be +/−10% of the stated value. For example, “about 1 kg” means from 0.90 kg to 1.1 kg.

The optically detected magnetic resonance (ODMR) measurement of NV center spins, which is the basis of diamond quantum sensing, involves intense laser irradiation and has inevitable phototoxicity to bio-samples. To overcome the bottleneck issue in diamond-based quantum biosensing—the phototoxicity, the damage caused by laser irradiation to bio-samples, light sheet microscopy (LSM) is applied to widefield optically detected magnetic resonance (ODMR). A light sheet is used to excite the nanodiamonds (NDs) in one plane and collect their ODMR spectra. Then the light sheet is scanned in the z direction. Three-dimensional imaging and high sensitivity (several K/\sqrt{Hz}) of simultaneous multi-point temperature measurement in 3D space are achieved.

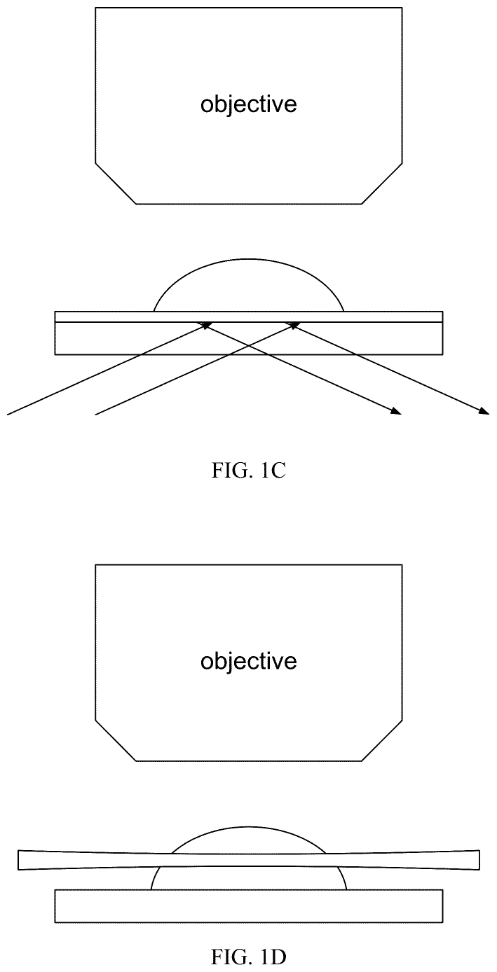

FIG. 2 shows the schematic of the LSM-ODMR setup. A light sheet is formed by a cylindrical lens CL and an objective O1. The sample is excited by the light sheet from one side. The fluorescence is collected by the collection objective O2 (which is perpendicular to O1). Different filters can be used to collect the fluorescence in certain wavelength range. A galvo mirror is used to scan the light sheet in the z direction to realize three-dimensional imaging and sensing. The sample is fixed on a 3D stage to adjust the position. The two optical elements O1 and CL are fixed on a 1D manual stage along the y direction (the propagation direction of the laser) so that the position of the light sheet along the y direction can be adjusted. Microwave is introduced by an antenna with frequency sweeping. An sCMOS camera is used to collect the fluorescence of nanodiamonds samples under each microwave frequency point with the same exposure time to get the ODMR signal. Through scanning the light sheet in the z direction, the ODMR spectra of the diamond samples in a 3D volume can be collected. The temperature sensitivity of nanodiamonds using LSM-ODMR system can reach several K/\sqrt{Hz}.

When compared to the conventional confocal ODMR, the LSM-ODMR system of the subject invention has higher sensing speed and lower phototoxicity to bio-samples. When compared to the conventional widefield ODMR, the LSM-ODMR system of the subject invention has lower phototoxicity to bio-samples and better signal to noise ratio. When compared to the conventional TIRF-based ODMR, the LSM-ODMR of the subject invention can sense much larger distance in the z direction such that 3D imaging can be realized.

### Materials and Methods

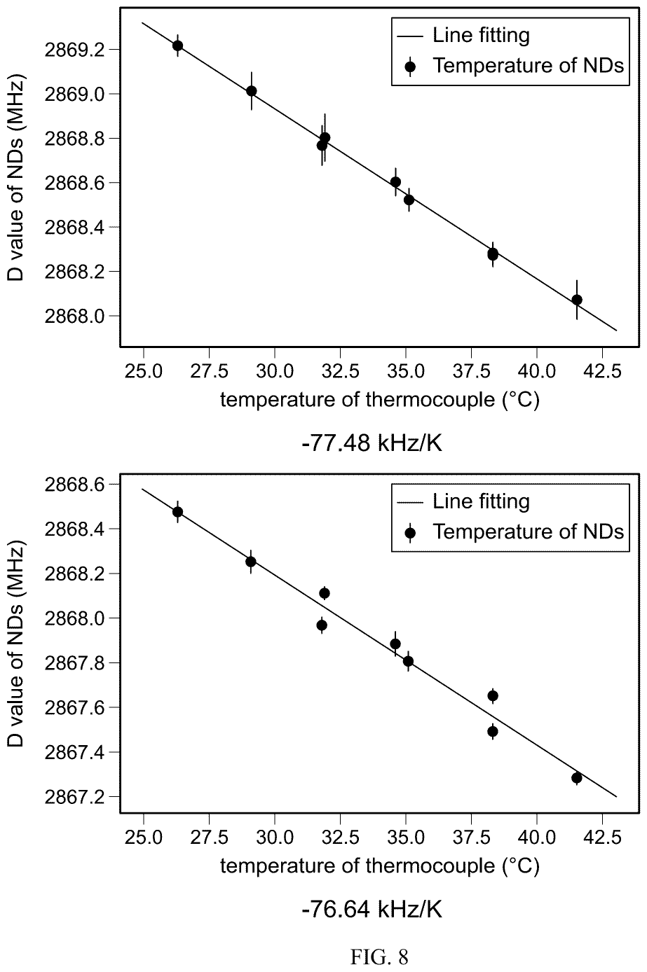

The temperature sensitivity of the LSM-ODMR system of the subject invention can reach several K/\sqrt{Hz}, for example, in a range between 2 K/\sqrt{Hz} and 11K/\sqrt{Hz} as shown in FIG. 7, which is comparable to the performance of the traditional widefield ODMR system. The method of measurements may be utilized within biological systems.

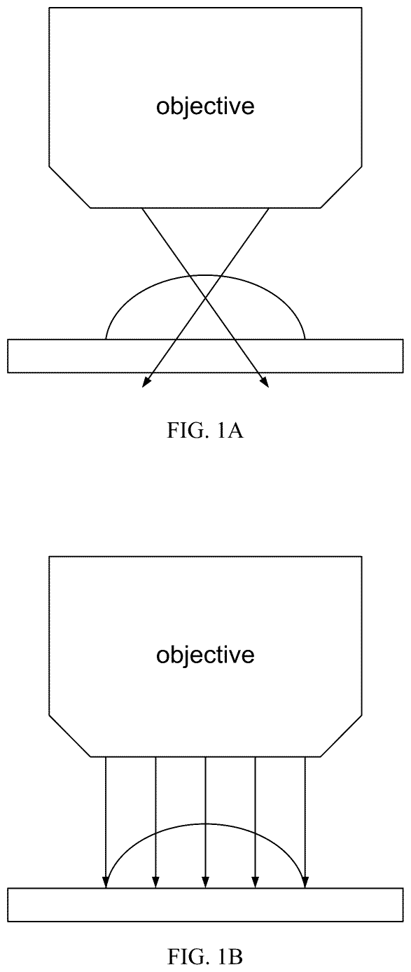

Referring to FIGS. 1A-1D, different excitation methods are compared. FIG. 1A shows the confocal microscopy. It uses point illumination and can only collect fluorescence within this point at a time. The imaging speed is significantly limited. Moreover, obtaining complete information of the sample (by scanning the focused point across the entire sample) needs a high laser dose.

FIG. 1B shows the widefield microscopy. The whole sample is exposed to laser, but only the sample layer on the focal plane of the objective can be detected. The laser dose is very high and the off-focus illumination and fluorescence are unavoidable.

FIG. 1C shows the TIRF mode microscopy. The phototoxicity is suppressed since the laser is confined within the thin evanescent-wave layer at the surface, but it can sense only one thin (about 100 nm) layer of samples close to the surface and is complicated by the interference of surface effects with the cellular functions.

FIG. 1D shows the LSM. It uses a light sheet to illustrate a thin layer of the sample perpendicular to the collection objective axis. It has fast imaging speed, low phototoxicity and is not confined to the evanescent wave layer in z direction.

FIG. 2 is a schematic representation of the LSM-ODMR setup of the subject invention. Laser 1 is a green laser with a wavelength of 532 nm, for exciting the NV centers in the sample. Laser 2 is a blue laser with a wavelength of 473 nm for exciting the fluorescent dye for imaging. A dichroic mirror is configured to overlap the two beams from the Laser 1 and Laser 2, respectively. After expanded by a couple of convex lenses (L₁ and L₂ for laser 1, L₃ and L₄ for laser 2) and passing an acousto-optic modulator (AOM, only for laser 1), the beam passes a cylindrical lens (CL) so that it is compressed in one direction (x) while remains in parallel to another direction (z). Then the beam passes the exciting objective O₁. After O₁, the beam is focused in the z direction and recovers in parallel to the x direction. Thus, a light sheet is formed around the focus of the exciting objective O₁. The sample is excited by the light sheet from one side. The fluorescence is collected by the collection objective O₂, which is perpendicular to O₁. Moreover, different filters can be configured before the sCMOS camera to collect the fluorescence in certain wavelength ranges. A galvo mirror is configured to scan the light sheet in the z direction to realize three-dimensional imaging. Further, the sample is fixed on a 3D stage being manually and piezoelectrically controlled with a manual adjustment range of about 20 mm and a piezoelectric control adjustment range of about 20 μm. The resolution can reach several nm. The two optical elements O₁ and CL are fixed on a 1-D manual stage along the y direction that is the propagation direction of the laser so the position of the light sheet in the y axis can be adjusted (the light sheet can be put in the middle of the field of view). Microwave from a signal generator is gated by an RF switch, amplified and then introduced to the sample by a copper line with frequency sweeping or at the resonant value. A sCMOS camera may be utilized to collect the fluorescence of diamond samples under each microwave frequency point with the same exposure time to get the ODMR signal. For LSM-T₁ relaxation measurement, a pulse streamer is employed to send pulse sequences to AOM, RF switch and the sCMOS camera.

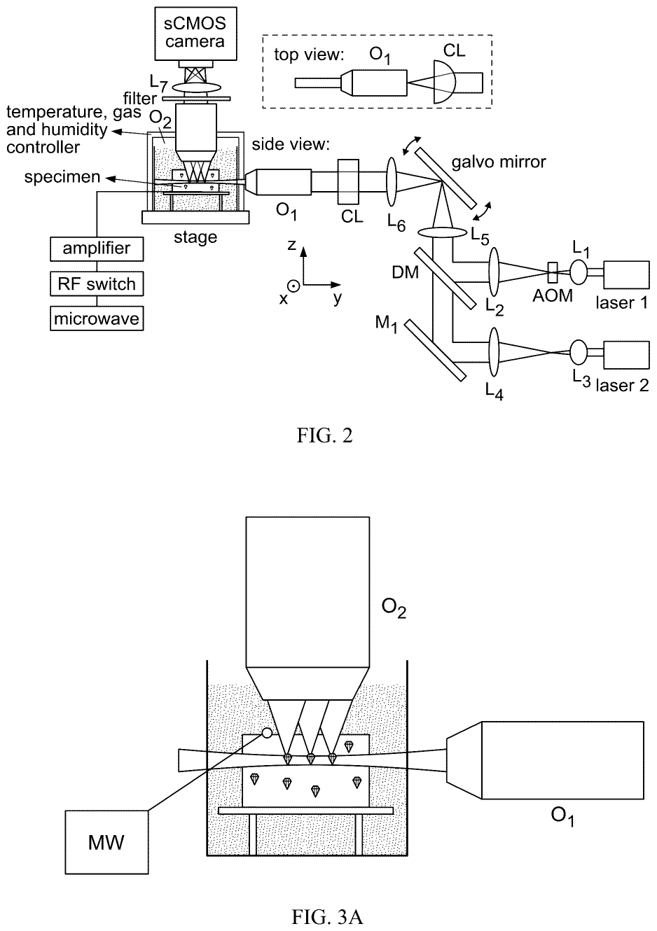

FIGS. 3A-3B show the sample holders. The LSM-ODMR sample is fixed in a small dish filled with water. The exciting objective lens O₁ is an air immersion objective lens with a working distance (WD) of 20.3 mm and a numerical aperture (NA) of 0.21. The detection objective O₂ is a water immersion objective (WD=2.8 mm, NA=1.0).

For those samples that have certain height, like bulk diamond sample and agarose sample, the coverslip could be put horizontally, as shown in FIG. 3A; while for those adherent samples, such as a coverslip with nanodiamonds dropped on it directly, and adherent cell samples, the exciting light will pass through the horizontal coverslip, which will cause aberration. To avoid the aberration, adherent samples are fixed with an angle. The designed angle is about 25° to the horizontal plane to avoid aberration while the detection objective is not influenced, as shown in FIG. 3B.

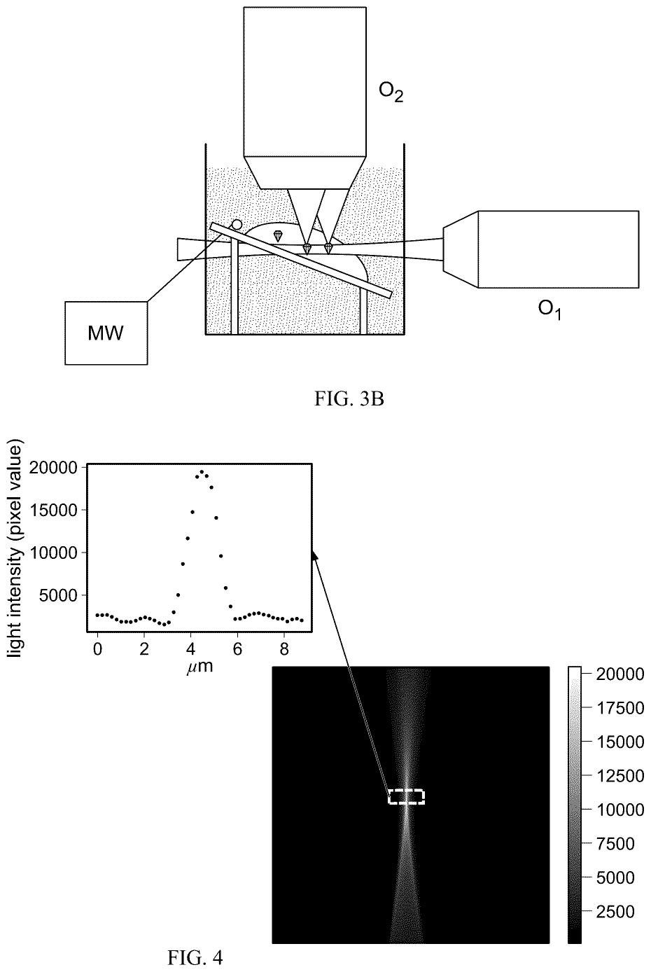

FIG. 4 is the visualization of the light sheet. A fluorescent dye, Rhodamine B, is used to visualize the generated light sheet. 0.1% Rhodamine B solution is excited by the light sheet. The fluorescence image is captured by the sCMOS camera. The color bar shows the relative fluorescence intensity (unit: pixel value).

A nanodiamond sample is used to measure the thickness of the light sheet. A single nanodiamond is scanned through the light sheet in z direction in small steps (0.2 μm step size in this case). Under each step, an image is captured by the sCMOS camera to record the fluorescence intensity. Before saturated, the fluorescence of a nanodiamond is proportional to the laser intensity. Hence, the fluorescence can be measured to represent the laser intensity distribution of the light sheet. The thickness of the light sheet is about 1.6 μm (full width at half maximum, FWHM). The Rayleigh length is about 16 μm.

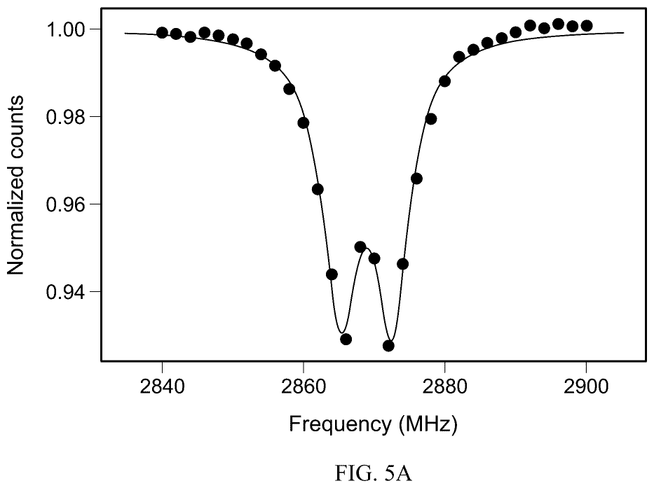

FIGS. 5A-5B show an exemplary LSM-ODMR spectrum. In particular, FIG. 5A is the normalized ODMR spectrum of an ND. The power density of the light sheet is about 20 μW/μm². The count rate is about 6,000 k counts per second. The ODMR signal is fitted with a two-peak Lorentzian curve. The contrasts of the two dips are 6.1% and 6.2%. The broadenings (FWHM) of the two dips are 6.2 MHz and 5.9 MHz. FIG. 5B is the measured temperature sensitivity of the ND in FIG. 5A. The horizontal coordinate is the square root of data collection frequency. The vertical coordinate is the standard deviation of the measured temperature. A 3 K/\sqrt{Hz} temperature sensitivity is achieved.

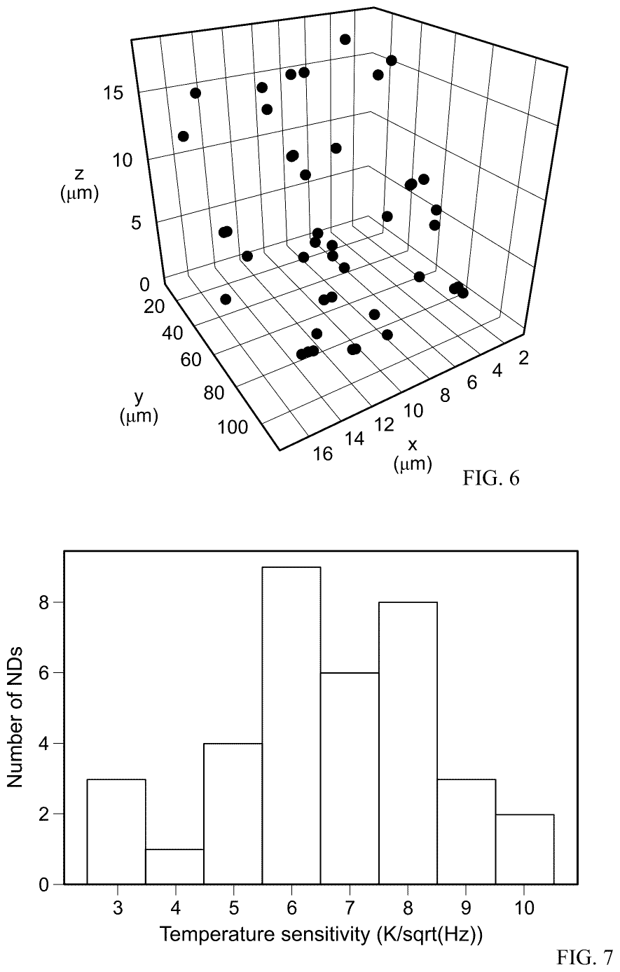

FIG. 6 is the 3D image of a ND+agarose sample. The ND samples are dispersed in the agarose. The dots represent NDs. The ability of 3D imaging and collecting ODMR with spatial information is shown. The laser propagates in the x direction. The sensing distance in the x direction is limited by the length of the light sheet. The laser is parallel in the y direction so a relatively large field of view in the y direction can be obtained. The light sheet is scanned in the z direction. The scanning range depends on the moving range of the galvo mirror and the piezo stage controlling of the collection objective, which is about 80 μm in the present setup.

FIG. 7 shows the temperature sensitivity distribution of the 36 NDs observed.

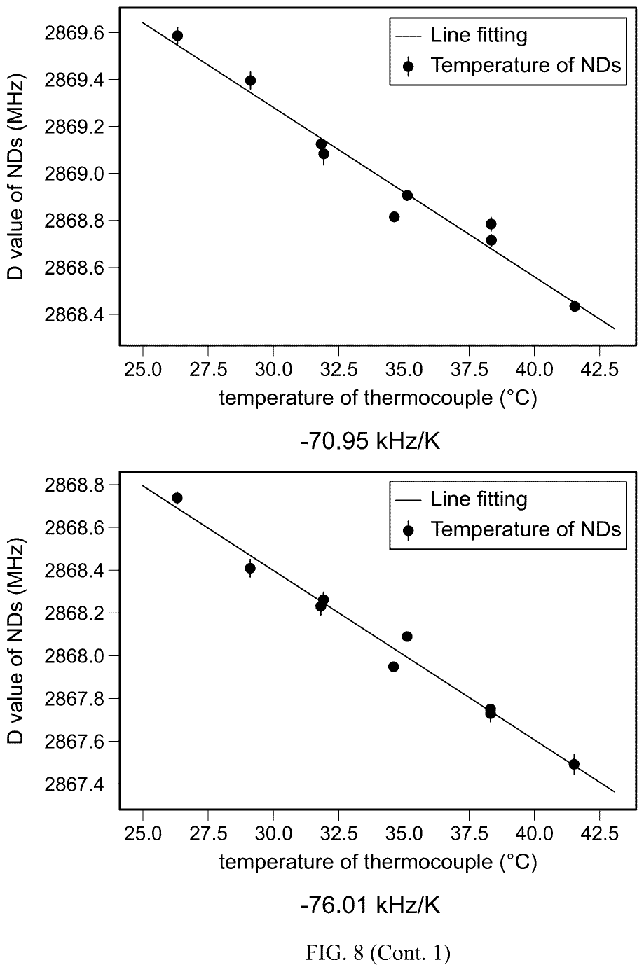

FIG. 8 shows certain dD/dT calibration results of the 36 NDs. The horizontal coordinate is the temperature measured by the thermocouple. The vertical coordinate is the zero-field splitting value D of the NDs. The error bars show the fitting errors. The dD/dT of each ND is fitted with a straight line. The slopes are shown under each inset.

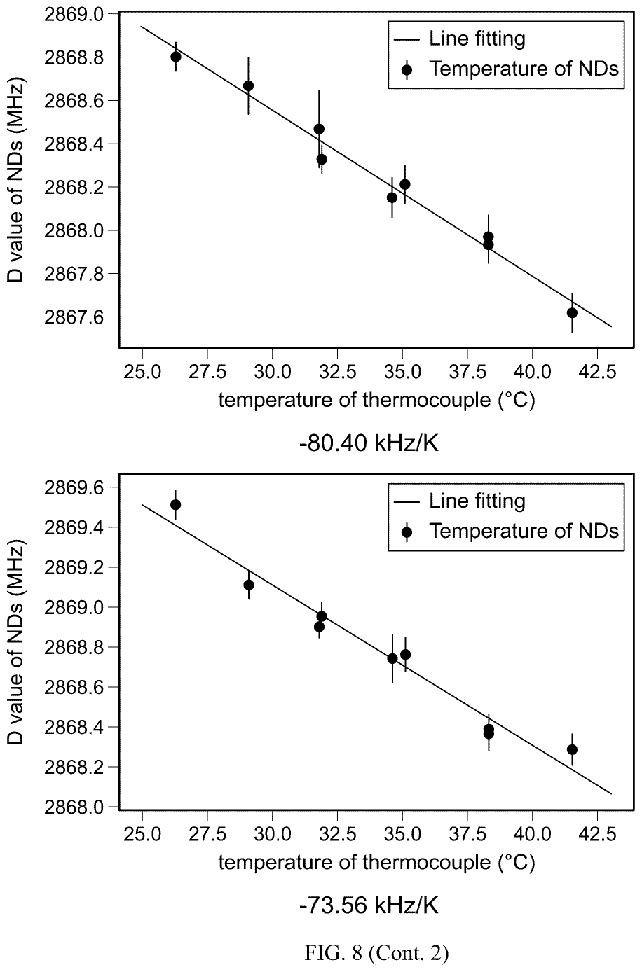

FIG. 9 is the dD/dT distribution of the NDs. The mean value is −76.2 kHz/K, which is close to literature [6].

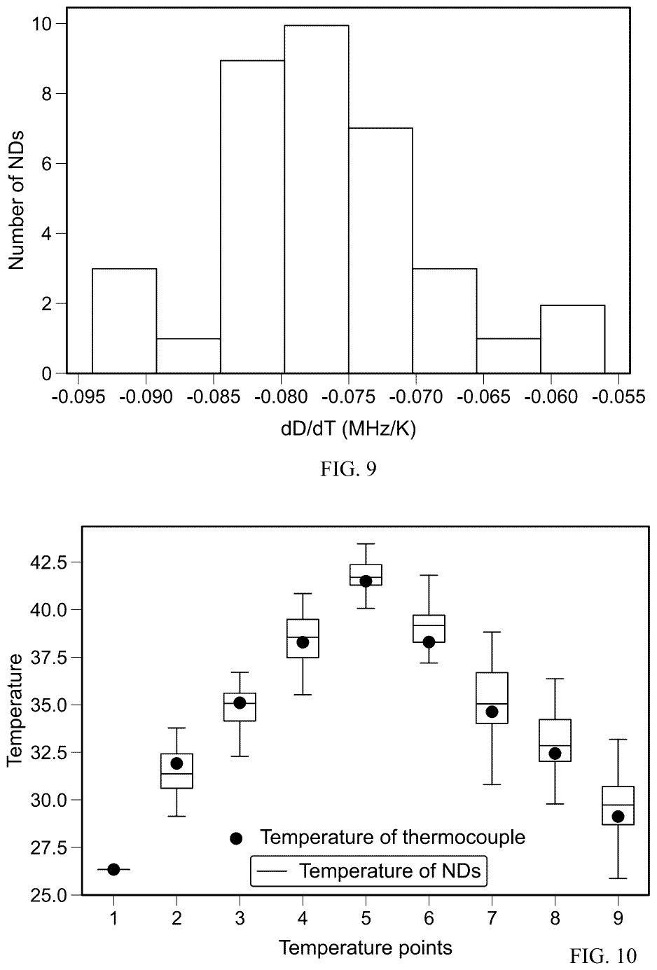

FIG. 10 shows the temperature measuring ability of the LSM-ODMR method. The temperature of the NDs embedded in the agarose sample is controlled by a temperature controller. The red dots show the set temperature points (enabled by a thermocouple). The box plots show the temperature measured by the NDs.

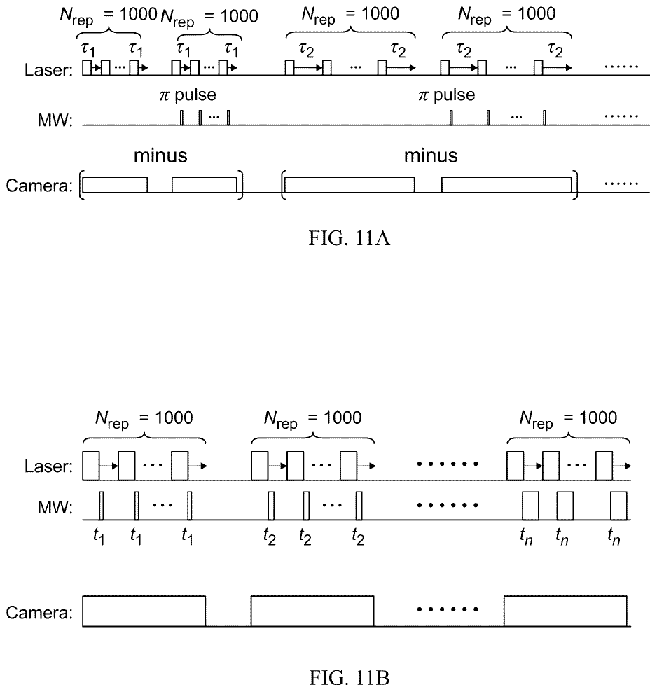

FIGS. 11A-11B show the pulse sequences used for LSM-T₁ measurement. The three lines from top to bottom are the pulse sequences sent to the AOM (laser), RF switch (microwave) and the sCMOS camera, respectively. The green, grey and red rectangles indicate that the laser, microwave and camera are on, respectively. FIG. 11A shows the sequences used for T₁ measurement. τₙ (n=1, 2, 3, . . . ) is the dark time. FIG. 11B shows the sequences used for Rabi oscillation.

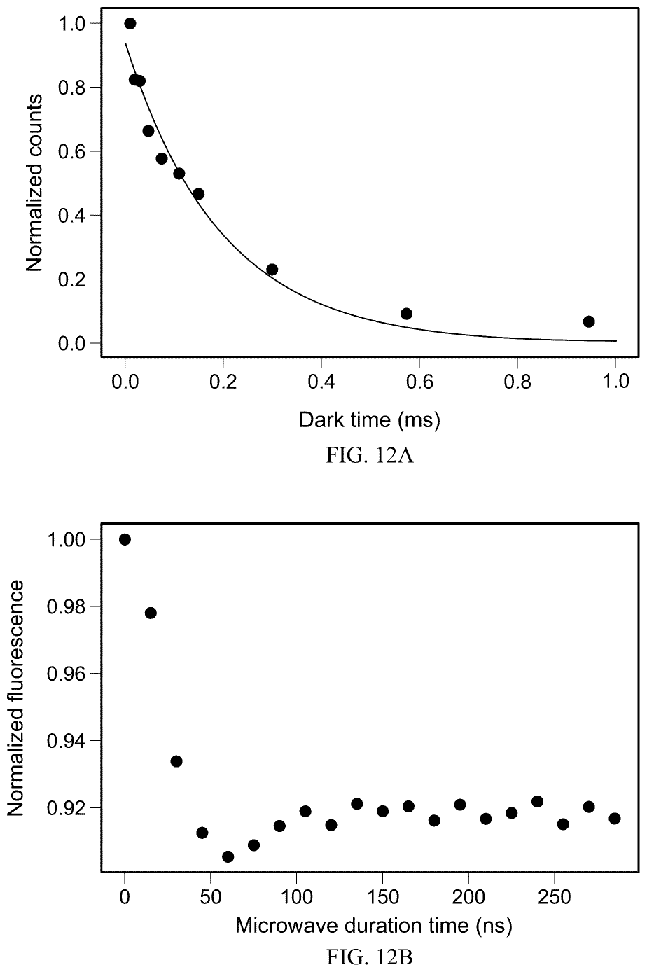

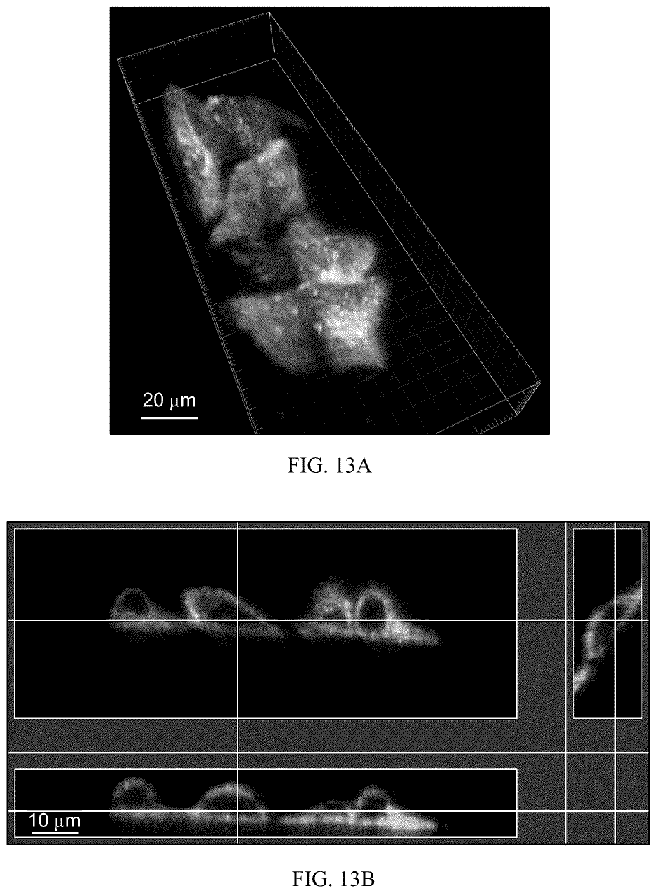

FIGS. 12A-12B show representative results of LSM-T₁ relaxation measurement. FIG. 12A shows a T₁ result of 196.2±24.7 μs fitted by an exponential decay function with a collection time of about 60 s. FIG. 12B shows a result of Rabi oscillation. The length of the π pulse is about 60 ns. Moreover, FIGS. 13A-13B show images of the Hela cells and NDs using LSM-ODMR setup. The Hela cells are transfected by CellMask Green dye. The green signal is the fluorescence from the transfected plasma membrane. The red signal is the fluorescence of NDs. FIG. 13A is the 3D image. FIG. 13B shows the x-y, x-z, y-z cross-sectional images.

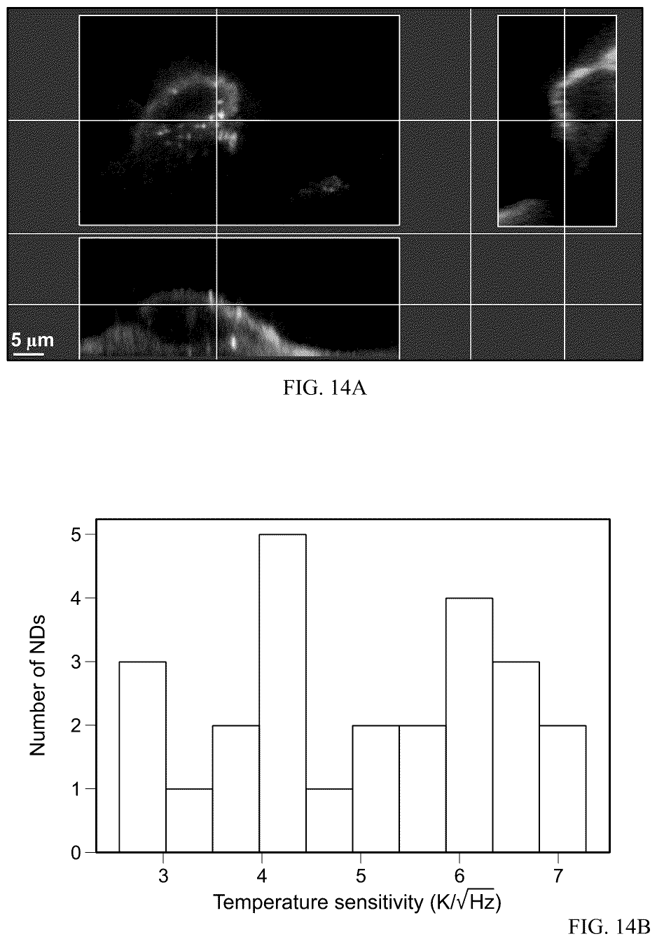

Further, FIGS. 14A-14B show the temperature sensitivity distribution of the nanodiamonds in a Hela cell using LSM-ODMR. In particular, FIG. 14A shows the image of the single Hela cell and the NDs and FIG. 14B shows the temperature sensitivity distribution of the NDs in FIG. 14A, which are in the order of K/\sqrt{Hz}.

### Test of the LSM-ODMR System

A 532 nm light sheet is used as the exciting light to carry out the widefield ODMR. The NDs sample is adjusted to the waist of the light sheet. A microwave with frequency sweeping is introduced by an antenna. The fluorescence is collected by a sCMOS camera. To reduce the laser dose, the laser power density is adjusted to about 20 μW/μm². The microwave power is adjusted to optimize the ODMR spectrum to reduce the linewidth and to enhance the contrast of the dips. The exposure time under each microwave frequency point is set to be 10 ms. After eliminating most of the vibrations in the light path, relatively high sensitivity of temperature measurement is achieved. Most of the nanodiamonds can reach a temperature sensitivity of a few K/\sqrt{Hz}. Further, FIG. 5A and FIG. 5B shows a representative ND with a temperature sensitivity of 3 K/\sqrt{Hz}, and FIG. 7 shows the distribution of the temperature sensitivity of the NDs embedded in agarose.

3D Imaging and Collecting ODMR Signals with Spatial Information

NDs embedded in agarose are used to demonstrate the ability of 3D imaging and collecting ODMR with spatial information. Agarose becomes liquid when it is heated to about 85-90° C. and becomes gel when it is cooled down to room temperature. 20 μg/ml ND aqueous solution and 2% agarose are mixed with equal proportion while heating. The agarose is gelled at room temperature to embed the NDs. The final mass fraction of agarose is 1%. The sample has a refractive index about 1.33, close to that of water (so it causes negligible aberration). The 3D distribution of the NDs is obtained by scanning the light sheet while collecting their ODMR spectra. The 3D distribution of NDs is shown in FIG. 6.

### Temperature Sensing Using LSM-ODMR

The energy difference between the ground state mₛ=0 and mₛ=±1 under zero field is D=2.87 GHz at room temperature, with temperature dependence dD/dT≈−74 kHz/K. Measuring D using ODMR of NDs offers a new approach in nano-thermometry.

To show the temperature sensing ability of the LSM-ODMR system, NDs are used to sense the temperature change of the system. A temperature controller is used to change the temperature of the sample and used NDs to detect the change. The agarose sample with NDs embedded is adopted. The NDs with relatively good temperature sensitivity are selected. The NDs with either low counts rate, small ODMR contrast or large ODMR broadening have relatively poor sensitivity and are not used. FIG. 7 shows the distribution of the temperature sensitivity of the selected NDs (36 NDs in total). Most of the NDs have a temperature sensitivity <10 K/\sqrt{Hz}.

A series of temperature points are set with a step of about 3° C. The temperatures are calibrated by a thermocouple inside the chamber. The heater is in the side walls and the lid of the chamber. Under each temperature point, the light sheet is scanned through the sample in the z direction. The ODMR spectra are collected with an integration time of 1.9 s on each frequency point (31 frequency points in total). FIG. 8 shows the D values fitted from the ODMR spectra against the set temperature. The error bars show the fitting errors of the double Lorentzian peak fitting. Each ND's dD/dT slope is fitted with a straight line. The dD/dT distribution is shown in FIG. 9, with a mean value of −76.2 kHz/K, which is consistent with literature [6].

FIG. 10 shows the temperature measured by the NDs. The temperature of NDs is deduced by the slope k₁ fitted from FIG. 8 and the zero-field splitting D value under the first temperature point (room temperature). The temperature measured by the ith ND at the jth temperature point is calculated as

$${T}_{i , j} = {T}_{i , 1} + \frac{{D}_{i , j} - {D}_{i , 1}}{{k}_{i}} .$$

The box plots show the middle, the first quartile, the third quartile, the minimum and the maximum values of the temperatures measured using the 36 NDs. The red dots show the set temperature points and the set temperatures are within the boxes.

### LSM-T₁ Measurement

FIGS. 11A-11B show the pulse sequences used for LSM-T₁ measurement. In particular, FIG. 11A shows the sequences used for T₁ measurement. The three lines from top to bottom are the pulse sequences sent to the AOM (laser), RF switch (microwave) and the sCMOS camera, respectively. τₙ (n=1, 2, 3, . . . ) is the dark time. The green, grey and red rectangles indicate that the laser, microwave and camera are on, respectively. The short laser pulse used to polarize and read out the NV center spins is set to 5 μs, then followed by a dark time τₙ. When measuring the relaxation process from mₛ=0 state, no microwave pulse is applied; when measuring the relaxation process from mₛ=±1 state, a π pulse of microwave is applied right after the laser pulse to drive the spin to mₛ=±1 state before relaxation. To sketch a T₁ curve of nanodiamonds (the T₁ time of nanodiamonds is usually in the order of several hundred microseconds), the dark time τₙ needs to range from the μs scale to the ms scale. For instance, the fluorescence detector is an sCMOS camera, whose shutter speed cannot reach the order of μs, and also to ensure enough fluorescence signal is collected in one frame, each of the dark time τₙ is repeated for Nᵣₑₚ times while the camera remains open (as illustrated in FIG. 11A). In this case, Nᵣₑₚ is set to 1000. As a result, the exposure time for each frame of the camera varies from milliseconds to seconds depending on the dark time. For every dark time, both relaxation processes from mₛ=0 and mₛ=±1 are measured. The signal is extracted by the difference of the fluorescence of mₛ=0 and mₛ=+1. A typical T₁ result of a nanodiamond using LSM-ODMR setup is shown in FIG. 12A. The collection time is about 60 s. The corresponding T₁ value is 196.2±24.7 μs fitted by an exponential decay function. For LSM-T₁ relaxation measurement, the T₁ values of the nanodiamonds in the FOV of the camera can be simultaneously measured.

The length of π pulse of microwave is determined by the Rabi measurement. The pulse sequences used is illustrated in FIG. 11B. A laser pulse of 5 μs is used to polarize the NV centers to mₛ=0, followed by a resonant microwave pulse with varying duration time tₙ (n=1, 2, 3, . . . ) to drive the spin between mₛ=0 and mₛ=±1 [34]. Then the same laser pulse is used to read out and reinitialize the electron spin. The fluorescence detector is the sCMOS camera. Similar to the T₁ measurement, each microwave duration time is repeated for Nᵣₑₚ times while the camera remains open. In this case, Nᵣₑₚ is set to 1000. Rabi measurement results are shown in FIG. 12B. the π pulse is approximately 60 ns. The microwave adopted is linearly polarized. The magnetic field part of the microwave can be decomposed into the \vec{B_{∥}} that is in parallel to the NV axis and the \vec{B_{⊥}} that is perpendicular to the NV axis. Only \vec{B_{⊥}} can drive the spin transitions. The Rabi frequency is proportional to the magnitude of \vec{B_{⊥}}. The angles between the magnetic field of microwave and the four NV axes in a nanodiamond are different, resulting in different \vec{B_{⊥}}. As a result, the Rabi oscillation without an external magnetic field is actually a superposition of the Rabi oscillations of the NV centers along the four possible axes. This explains the rapid decay of the oscillation observed in FIG. 12B. In experiment, the lengths of the π pulse of different nanodiamonds show little variation, if the distances between the nanodiamonds and the microwave line do not vary too much. Thus, a π pulse of fixed length can be used.

### LSM Cell Images

The LSM-ODMR system configures a suitable growth environment for cells and can realize rapid 3D cell imaging and intracellular ODMR experiments.

A 10×10 mm coverslip is ultrasonically cleaned with ethanol, then cleaned with plasma. Then we use polydimethylsiloxane (PDMS) to attach a copper wire to the coverslip to deliver microwave. Then the coverslip is put into a cell culture dish. Hela cells are seeded at an appropriate density with Dulbecco's modified Eagle's medium (DMEM, Gibco), supplemented with 10% Fetal Bovine Serum (FBS), 0.1 g/L streptomycin sulfate, and 0.06 g/L penicillin G [20]. The cells are incubated at 37° C. with humidity and CO₂ level controlled to let them adhere on the coverslip and grow to an appropriate density. Then the cells are incubated with 2 μg/ml NDs in DMEM for 1 hour and then wash away the NDs and add fresh medium. The cells are further incubated overnight. Then the cells are stained with dyes for imaging and helping determine the relative position of the NDs to the cells.

The coverslip is transferred into the LSM-ODMR sample holder as shown in FIG. 3B and mounted to the LSM-ODMR setup. A controller (for example, from Okolab) is configured to control the temperature, humidity and CO₂ level, as illustrated in FIG. 2.

A three-dimensional image of Hela cells and intracellular NDs is shown in FIG. 13A. The Hela cells are transfected by CellMask Green dye. The green signal is the fluorescence from the transfected plasma membrane. The red signal is the fluorescence of NDs. FIG. 13B shows the x-y, x-z, y-z cross-sectional images of the Hela cells and NDs. Different filters before the camera are selected to distinguish the fluorescence of the dye and the NDs. For the cell image, the exciting light is the 473 nm laser, and the collected fluorescence wavelength range is 520-605 nm; for the ND image, the exciting light is the 532 nm laser, and the collected fluorescence wavelength is above 650 nm. In each frame, a 2D image is captured. Then the sample is scanned in the z direction by moving the stage or scanning the galvo mirror to form a 3D image.

### ODMR Result of Intracellular NDs

ODMR experiment with high sensitivity has been carried out in Hela cells using LSM-ODMR setup. FIGS. 14A and 14B show the temperature sensitivity distribution of the nanodiamonds in a Hela cell. In particular, FIG. 14A shows the image of the single Hela cell and the NDs and FIG. 14B shows the temperature sensitivity distribution of the NDs in FIG. 14A. The power density of 532 nm laser is set to about 15 μW/μm². After the experiment, HeLa cells maintained their normal adherent morphology and membrane integrity.

According to the embodiments of the subject invention, the LSM-ODMR system provides a method for fast, low phototoxicity, and high sensitivity measurement for bio-sensing.

To overcome the bottleneck issue in diamond-based biosensing—the phototoxicity, light sheet microscopy (LSM) is applied to widefield optically detected magnetic resonance (ODMR). A light sheet is used to excite nanodiamonds (NDs) in one plane and collect their ODMR spectra. Then the light sheet is scanned in z direction. Three-dimensional imaging and high sensitivity (several K/\sqrt{Hz}) of multi-location temperature measurement in 3D space are achieved. Relatively fast LSM-T₁ measurement is also shown. For bio-sensing, the 3D image of Hela cells and intracellular NDs is captured by the LSM-ODMR system. The temperature sensitivity of intracellular NDs can also reach the order of K/\sqrt{Hz}.

The LSM-ODMR system of the subject invention, by solving the bottleneck issue of phototoxicity in bio-applications, provides a new approach to studying machineries in bio-samples, with high time and spatial resolution, capability of three-dimensional and even four dimensional (space and time) imaging, and multi-modal sensitivity. The LSM-ODMR system can be applied to bio-sensing applications such as nano thermometry in bio-systems, intracellular orientation tracking, detecting paramagnetic species and sub-cellular magnetic imaging with much lower phototoxicity (compared to widefield ODMR) and much larger sensing distance, as compared to ODMR based on the total internal reflection fluorescence (TIRF) microscopy.

Hence, the LSM-ODMR system of the subject invention presents avenues for rapid, low phototoxicity, and nanoscale resolution bio-sensing. It proves valuable in detecting biological processes due to the sensitivity of ODMR spectra to various parameters such as temperature, magnetic field, and pressure. Additionally, the LSM minimizes perturbations to the bio-samples caused by laser irradiation.

Further, the LSM-ODMR system of the subject invention offers advantages including, but not limited to, higher sensing speed and lower phototoxicity than confocal ODMR since confocal scans the sample in a point-by-point way; lower phototoxicity and better signal to noise ratio than the conventional widefield ODMR since only the detected layer of sample is illuminated at a time; capabilities to sense larger distance in z direction compared with TIRF mode ODMR since the light sheet is movable along the z-axis and is not confined in the thin evanescent-wave layer at the surface.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### EXEMPLARY EMBODIMENTS

Embodiment 1. A system based on light sheet microscopy (LSM) and optically detected magnetic resonance (ODMR), comprising:
a cylindrical lens CL;
an objective lens O1 combined with the cylindrical lens CL to generate a light sheet for exciting a sample from one side;
a collection objective lens O2 for collecting fluorescence generated by the sample;
a galvo mirror configured to scan the light sheet in a first direction to realize three-dimensional (3D) imaging and sensing;
an antenna configured to introduce microwave with frequency sweeping to the sample; and
an image sensor configured to collect fluorescence of the sample under each microwave frequency point with same exposure time to obtain ODMR signals;
wherein the sample is fixed on a 3D stage and a position of the sample is adjustable, and
wherein the cylindrical lens CL and the objective lens O1 are fixed on a stage along a second direction that is a propagation direction of a laser beam such that a position of the light sheet is adjustable along the second direction.

Embodiment 2. The system of embodiment 1, wherein the collection objective lens O2 is disposed in a direction perpendicular to the objective lens O1.

Embodiment 3. The system of embodiment 1, further comprising one or more filters configured to collect the fluorescence in a predetermined wavelength range.

Embodiment 4. The system of embodiment 1, wherein the image sensor is a sCMOS camera.

Embodiment 5. The system of embodiment 1, wherein the sample is a plurality of nanodiamonds.

Embodiment 6. The system of embodiment 1, wherein by scanning the light sheet in the first direction, ODMR spectra of the sample in a 3D volume are collected.

Embodiment 7. The system of embodiment 1, wherein a temperature sensitivity of the sample is determined and is on a scale of K/\sqrt{Hz}.

Embodiment 8. The system of embodiment 1, wherein the objective lens O1 is an air immersion objective lens.

Embodiment 9. The system of embodiment 8, wherein the objective lens O1 has a working distance (WD) of 20.3 mm and a numerical aperture (NA) of 0.21.

Embodiment 10. The system of embodiment 1, wherein the collection objective lens O2 is a water immersion objective lens having a working distance of 2.8 mm and a numerical aperture of 1.0.

Embodiment 11. The system of embodiment 1, further comprising a first laser emitting a first laser beam for exciting the NV centers in the sample.

Embodiment 12. The system of embodiment 11, further comprising a second laser emitting a second laser beam for exciting fluorescent dye for imaging.

Embodiment 13. The system of embodiment 12, further comprising a dichroic mirror configured to overlap the first and second laser beams.

Embodiment 14. The system of embodiment 13, wherein the first laser beam is expanded by first and second convex lenses L₁ and L₂.

Embodiment 15. The system of embodiment 14, wherein the second laser beam is expanded by third and fourth convex lenses L₃ and L₄.

Embodiment 16. The system of embodiment 15, wherein the first laser beam passes through an acousto-optic modulator AOM disposed between the first and second convex lenses L₁ and L₂.

Embodiment 17. The system of embodiment 16, wherein the beam passes the cylindrical lens CL such that it is compressed in one direction x while remains in parallel to another direction z.

Embodiment 18. The system of embodiment 11, wherein the first laser beam is a green laser beam with a wavelength of about 532 nm.

Embodiment 19. The system of embodiment 12, wherein the second laser beam is a blue laser with a wavelength of about 473 nm.

Embodiment 20. The system of embodiment 1, further comprising a controller configured to control temperature, humidity and CO₂ level of the sample.

Embodiment 21. The system of embodiment 1, wherein the microwave is gated by an RF switch, then amplified by an amplifier, and then introduced to the sample by a copper line with frequency sweeping or at a resonant value.

Embodiment 22. A method based on light sheet microscopy (LSM) and optically
detected magnetic resonance (ODMR), comprising:
exciting a sample from one side, by a light sheet generated by a cylindrical lens CL and an objective lens O1;
collecting, by a collection objective lens O2, fluorescence generated by the sample;
scanning, by a galvo mirror, the light sheet in a first direction to realize three-dimensional (3D) imaging and sensing;
introducing, by an antenna, microwave with frequency sweeping to the sample; and
collecting by an image sensor, the fluorescence of the sample under each microwave frequency point with same exposure time to obtain ODMR signals;
wherein the sample is fixed on a 3D stage and a position of the sample is adjustable, and
wherein the cylindrical lens CL and the objective lens O1 are fixed on a stage along a second direction which is a propagation direction of a laser beam such that a position of the light sheet is adjustable along the second direction.

Embodiment 23. The method of embodiment 22, wherein the collection objective lens O2 is disposed in a direction perpendicular to the objective lens O1.

Embodiment 24. The method of embodiment 22, further comprising providing one or more filters for collecting the fluorescence in a predetermined wavelength range.

Embodiment 25. The method of embodiment 22, wherein the image sensor is a sCMOS camera.

Embodiment 26. The method of embodiment 22, wherein the sample is a plurality of nanodiamonds.

Embodiment 27. The method of embodiment 22, wherein by scanning the light sheet in the first direction, ODMR spectra of the sample in a 3D volume are collected.

Embodiment 28. The method of embodiment 22, wherein a temperature sensitivity of the sample is determined and is on a scale of K/\sqrt{Hz}.

Embodiment 29. The method of embodiment 22, wherein the objective lens O₁ is an air immersion objective lens.

Embodiment 30. The method of embodiment 29, wherein the objective lens O₁ has a working distance (WD) of 20.3 mm and a numerical aperture (NA) of 0.21.

Embodiment 31. The method of embodiment 22, wherein the collection objective lens O2 is a water immersion objective lens having a working distance of 2.8 mm and a numerical aperture of 1.0.

Embodiment 32. The method of embodiment 22, further comprising providing a first laser beam for exciting the NV centers in the sample.

Embodiment 33. The method of embodiment 32, further comprising providing a second laser beam for exciting fluorescent dye for imaging.

Embodiment 34. The method of embodiment 33, further comprising providing a dichroic mirror configured to overlap the first and second laser beams.

Embodiment 35. The method of embodiment 34, wherein the first laser beam is expanded by first and second convex lenses L₁ and L₂.

Embodiment 36. The method of embodiment 35, wherein the second laser beam is expanded by third and fourth convex lenses L₃ and L₄.

Embodiment 37. The method of embodiment 36, wherein the first laser beam passes through an acousto-optic modulator AOM disposed between the first and second convex lenses L₁ and L₂.

Embodiment 38. The method of embodiment 37, wherein the beam passes the cylindrical lens CL such that it is compressed in one direction x while remains in parallel to another direction z.

Embodiment 39. The method of embodiment 32, wherein the first laser beam is a green laser beam with a wavelength of about 532 nm.

Embodiment 40. The method of embodiment 33, wherein the second laser beam is a blue laser with a wavelength of about 473 nm.

Embodiment 41. The method of embodiment 22, further comprising configuring a controller to control temperature, humidity and/or CO₂ level of the sample.

Embodiment 42. The method of embodiment 22, wherein the microwave is gated by an RF switch, then amplified by an amplifier, and then introduced to the sample by a copper line with frequency sweeping or at a resonant value.

### REFERENCES

[1]. Yu, Shu-Jung, et al. “Bright fluorescent nanodiamonds: no photobleaching and low cytotoxicity.” Journal of the American Chemical Society 127.50 (2005): 17604-17605.
[2]. Fu, Chi-Cheng, et al. “Characterization and application of single fluorescent nanodiamonds as cellular biomarkers.” Proceedings of the National Academy of Sciences 104.3 (2007): 727-732.
[3]. Kucsko, Georg, et al. “Nanometre-scale thermometry in a living cell.” Nature 500.7460 (2013): 54-58.
[4]. Gruber, A., et al. “Scanning confocal optical microscopy and magnetic resonance on single defect centers.” Science 276.5321 (1997): 2012-2014.
[5]. Taylor, Jacob M., et al. “High-sensitivity diamond magnetometer with nanoscale resolution.” Nature Physics 4.10 (2008): 810-816.
[6]. Acosta, Victor M., et al. “Temperature dependence of the nitrogen-vacancy magnetic resonance in diamond.” Physical review letters 104.7 (2010): 070801.
[7]. Neumann, Philipp, et al. “High-precision nanoscale temperature sensing using single defects in diamond.” Nano letters 13.6 (2013): 2738-2742.
[8]. Dolde, Florian, et al. “Electric-field sensing using single diamond spins.” Nature Physics 7.6 (2011): 459-463.
[9]. Doherty, Marcus W., et al. “Electronic properties and metrology applications of the diamond NV⁻ center under pressure.” Physical review letters 112.4 (2014): 047601.
[10]. Doherty, Marcus W., et al. “The nitrogen-vacancy colour centre in diamond.” Physics Reports 528.1 (2013): 1-45.
[11]. Nie, L., et al. “Quantum monitoring of cellular metabolic activities in single mitochondria.” Science advances 7.21 (2021): eabf0573.
[12]. Tetienne, J-P., et al. “Spin relaxometry of single nitrogen-vacancy defects in diamond nanocrystals for magnetic noise sensing.” Physical Review B-Condensed Matter and Materials Physics 87.23 (2013): 235436.
[13]. Sushkov, A. O., et al. “All-optical sensing of a single-molecule electron spin.” Nano letters 14.11 (2014): 6443-6448.
[14]. Iyer, Shiva, et al. “Optically-trapped-nanodiamond relaxometric detection of nanomolar paramagnetic spins in aqueous environments.” Physical Review Applied 22.6 (2024): 064076.
[15]. Vaijayanthimala, V., et al. “The long-term stability and biocompatibility of fluorescent nanodiamond as an in vivo contrast agent.” Biomaterials 33.31 (2012): 7794-7802.
[16]. Simpson, David A., et al. “Non-neurotoxic nanodiamond probes for intraneuronal temperature mapping.” ACS nano 11.12 (2017): 12077-12086.
[17]. Fujiwara, Masazumi, et al. “Real-time nanodiamond thermometry probing in vivo thermogenic responses.” Science advances 6.37 (2020): eaba9636.
[18]. Choi, Joonhee, et al. “Probing and manipulating embryogenesis via nanoscale thermometry and temperature control.” Proceedings of the National Academy of Sciences 117.26 (2020): 14636-14641.
[19]. McGuinness, Liam P., et al. “Quantum measurement and orientation tracking of fluorescent nanodiamonds inside living cells.” Nature nanotechnology 6.6 (2011): 358-363.
[20]. Feng, Xi, et al. “Association of nanodiamond rotation dynamics with cell activities by translation-rotation tracking.” Nano Letters 21.8 (2021): 3393-3400.
[21]. Le Sage, David, et al. “Optical magnetic imaging of living cells.” Nature 496.7446 (2013): 486-489.
[22]. Laissue, P. Philippe, et al. “Assessing phototoxicity in live fluorescence imaging.” Nature methods 14.7 (2017): 657-661.
[23]. Thannickal, Victor J., and Barry L. Fanburg. “Reactive oxygen species in cell signaling.” American Journal of Physiology-Lung Cellular and Molecular Physiology 279.6 (2000): L1005-L1028.
[24]. Sinha, Rajeshwar P., and Donat-P. Hader. “UV-induced DNA damage and repair: a review.” Photochemical & Photobiological Sciences 1.4 (2002): 225-236.
[25]. Schönle, Andreas, and Stefan W. Hell. “Heating by absorption in the focus of an objective lens.” Optics letters 23.5 (1998): 325-327.
[26]. Axelrod, Daniel. “Cell-substrate contacts illuminated by total internal reflection fluorescence.” The Journal of cell biology 89.1 (1981): 141-145.
[27]. Axelrod, Daniel. “Total internal reflection fluorescence microscopy in cell biology.” Traffic 2.11 (2001): 764-774.
[28]. Huisken, Jan, et al. “Optical sectioning deep inside live embryos by selective plane illumination microscopy.” Science 305.5686 (2004): 1007-1009.
[29]. Reynaud, Emmanuel G., et al. “Light sheet-based fluorescence microscopy: more dimensions, more photons, and less photodamage.” HFSP journal 2.5 (2008): 266-275.
[30]. Reynaud, Emmanuel G., et al. “Guide to light-sheet microscopy for adventurous biologists.” Nature methods 12.1 (2015): 30-34.
[31]. Olarte, Omar E., et al. “Light-sheet microscopy: a tutorial.” Advances in Optics and Photonics 10.1 (2018): 111-179.
[32]. Gao, Liang, et al. “3D live fluorescence imaging of cellular dynamics using Bessel beam plane illumination microscopy.” Nature protocols 9.5 (2014): 1083-1101.
[33]. Yang, Zhengyi, et al. “A compact Airy beam light sheet microscope with a tilted cylindrical lens.” Biomedical optics express 5.10 (2014): 3434-3442.
[34]. Scully, Marlan O., and M. Suhail Zubairy. Quantum optics. Cambridge university press, 1997.

1. A system based on light sheet microscopy (LSM) and optically detected magnetic resonance (ODMR), comprising:
a cylindrical lens CL;
an objective lens O1 combined with the cylindrical lens CL and configured to generate a light sheet for exciting a sample from one side;
a collection objective lens O2 configured to collect fluorescence generated by the sample;
a galvo mirror configured to scan the light sheet in a first direction to realize three-dimensional (3D) imaging and sensing;
an antenna configured to introduce microwave with frequency sweeping to the sample; and
an image sensor configured to collect fluorescence of the sample under each microwave frequency point with a same exposure time to obtain ODMR signals;
wherein the sample is fixed on a 3D stage and a position of the sample is adjustable, and
wherein the cylindrical lens CL and the objective lens O1 are fixed on a stage along a second direction that is a propagation direction of a laser beam such that a position of the light sheet is adjustable along the second direction.

2. The system of claim 1, wherein the collection objective lens O2 is disposed in a direction perpendicular to the objective lens O1.

3. The system of claim 1, further comprising one or more filters configured to collect the fluorescence in a predetermined wavelength range.

4. The system of claim 1, wherein the image sensor is a sCMOS camera.

5. The system of claim 1, wherein the sample is a plurality of nanodiamonds.

6. The system of claim 1, wherein by scanning the light sheet in the first direction, ODMR spectra of the sample in a 3D volume are collected.

7. The system of claim 1, wherein a temperature sensitivity of the sample is determined and is on a scale of K/\sqrt{Hz}.

8. The system of claim 1, wherein the objective lens O₁ is an air immersion objective lens.

9. The system of claim 8, wherein the objective lens O₁ has a working distance (WD) of 20.3 mm and a numerical aperture (NA) of 0.21.

10. The system of claim 1, wherein the collection objective lens O2 is a water immersion objective lens having a working distance of 2.8 mm and a numerical aperture of 1.0.

11. The system of claim 1, further comprising a first laser configured to emit a first laser beam for exciting the NV centers in the sample.

12. The system of claim 11, further comprising a second laser configured to emit a second laser beam for exciting fluorescent dye for imaging.

13. The system of claim 12, further comprising a dichroic mirror configured to overlap the first and second laser beams.

14. The system of claim 13, wherein the first laser beam is expanded by first and second convex lenses L₁ and L₂.

15. The system of claim 14, wherein the second laser beam is expanded by third and fourth convex lenses L₃ and L₄.

16. The system of claim 15, wherein the first laser beam passes through an acousto-optic modulator AOM disposed between the first and second convex lenses L₁ and L₂.

17. The system of claim 16, wherein the first laser beam passes the cylindrical lens CL such that it is compressed in one direction x while remains in parallel to another direction z.

18. The system of claim 11, wherein the first laser beam is a green laser beam with a wavelength of about 532 nm.

19. The system of claim 12, wherein the second laser beam is a blue laser with a wavelength of about 473 nm.

20. The system of claim 1, further comprising a controller configured to control temperature, humidity, and CO₂ level of the sample.

21. The system of claim 1, wherein the microwave is gated by an RF switch, then amplified by an amplifier, and then introduced to the sample by a copper line with frequency sweeping or at a resonant value.

22. A method based on light sheet microscopy (LSM) and optically detected magnetic resonance (ODMR), comprising:
exciting a sample from one side, by a light sheet generated by a cylindrical lens CL and an objective lens O1;
collecting, by a collection objective lens O2, fluorescence generated by the sample;
scanning, by a galvo mirror, the light sheet in a first direction to realize three-dimensional (3D) imaging and sensing;
introducing, by an antenna, microwave with frequency sweeping to the sample; and
collecting by an image sensor, the fluorescence of the sample under each microwave frequency point with a same exposure time to obtain ODMR signals;
wherein the sample is fixed on a 3D stage and a position of the sample is adjustable, and
wherein the cylindrical lens CL and the objective lens O1 are fixed on a stage along a second direction that is a propagation direction of a laser beam such that a position of the light sheet is adjustable along the second direction.

23. The method of claim 22, wherein the collection objective lens O2 is disposed in a direction perpendicular to the objective lens O1.

24. The method of claim 22, further comprising providing one or more filters configured to collect the fluorescence in a predetermined wavelength range.

25. The method of claim 22, wherein the image sensor is a sCMOS camera.

26. The method of claim 22, wherein the sample is a plurality of nanodiamonds.

27. The method of claim 22, wherein by scanning the light sheet in the first direction, ODMR spectra of the sample in a 3D volume are collected.

28. The method of claim 22, wherein a temperature sensitivity of the sample is determined and is on a scale of K/\sqrt{Hz}.

29. The method of claim 22, wherein the objective lens O₁ is an air immersion objective lens.

30. The method of claim 29, wherein the objective lens O₁ has a working distance (WD) of 20.3 mm and a numerical aperture (NA) of 0.21.

31. The method of claim 22, wherein the collection objective lens O2 is a water immersion objective lens having a working distance of 2.8 mm and a numerical aperture of 1.0.

32. The method of claim 22, further comprising providing a first laser beam for exciting the NV centers in the sample.

33. The method of claim 32, further comprising providing a second laser beam for exciting fluorescent dye for imaging.

34. The method of claim 33, further comprising providing a dichroic mirror configured to overlap the first and second laser beams.

35. The method of claim 34, wherein the first laser beam is expanded by first and second convex lenses L₁ and L₂.

36. The method of claim 35, wherein the second laser beam is expanded by third and fourth convex lenses L₃ and L₄.

37. The method of claim 36, wherein the first laser beam passes through an acousto-optic modulator AOM disposed between the first and second convex lenses L₁ and L₂.

38. The method of claim 37, wherein the first laser beam passes the cylindrical lens CL such that it is compressed in one direction x while remains in parallel to another direction z.

39. The method of claim 32, wherein the first laser beam is a green laser beam with a wavelength of about 532 nm.

40. The method of claim 33, wherein the second laser beam is a blue laser beam with a wavelength of about 473 nm.

41. The method of claim 22, further comprising configuring a controller to control temperature, humidity, and/or CO₂ level of the sample.

42. The method of claim 22, wherein the microwave is gated by an RF switch, then amplified by an amplifier, and then introduced to the sample by a copper line with frequency sweeping or at a resonant value.
